(19) United States
(12) Patent Application Publication
(10) Pub. No.: US 2016/0374659 A1
(43) Pub. Date: Dec. 29, 2016

(54) INTERVERTEBRAL DISTRACTING PORTAL

(71) Applicant: STERLING THERAPEUTICS, LLC, COLTS NECK, NJ (US)

(72) Inventor: GORDON D. DONALD, OCEANPORT, NJ (US)

(21) Appl. No.: 15/191,097

(22) Filed: Jun. 23, 2016

Related U.S. Application Data
(60) Provisional application No. 62/183,412, filed on Jun. 23, 2015.

(51) Int. Cl. A61B 17/02; A61B 1/313; A61B 1/00

(52) U.S. Cl. CPC A61B 17/025; A61B 17/0206; A61B 1/00154; A61B 1/3135; A61B 17/0218; A61B 2017/0256; A61B 2017/00261

(57) ABSTRACT

A distracting portal device for use between two adjacent vertebrae during an intervertebral surgical procedure. The distracting portal device is a three-dimensional object having a two-dimensional cross-section extended into a third dimension, where the cross-section has a shape which defines one or two portal openings. Each portal opening creates a space between the vertebrae into which a surgeon has clear access to perform the surgical procedure, such as a discectomy. Opposing top and bottom surfaces of the portal device are configured for contact with and distraction of the two adjacent vertebrae. The portal device is selected from a family having a single portal opening and having a cross-section resembling one of the letters “C”, “O” or “U”, or a family having two portal openings and having a cross-section resembling one of the letters “H” or “I”.

### CROSS-REFERENCE TO RELATED APPLICATIONS

This application claims the benefit of the priority date of U.S. Provisional Patent Application Ser. No. 62/183,412, titled, Intervertebral Distracting Portal, filed Jun. 23, 2015.

### BACKGROUND OF THE INVENTION

Field of the Invention

The invention relates to the field of interbody distractors, and more specifically, to intervertebral distractors providing surgical portal access to a disc while maintaining distraction.

Description of the Related Art

Intervertebral surgical techniques employ standard wedge distractors (trials) to mobilize the disc space. However, the disc space collapses when the distractor is removed to perform further discectomy. Moving instruments in and out of the area increases risk of damage to adjacent structures and dramatically decreases the efficiency of the procedure.

### SUMMARY OF THE INVENTION

In one or more embodiments, intervertebral distractor devices are disclosed which provide surgical portal access to a disc or vertebral motion segments which may span one or more disc(s) and vertebral body(s) while maintaining distraction. The disclosed intervertebral distractors provide access to work in and visualize disc space and/or spinal cavities, whether anatomic or surgically created. The devices improve discectomy by maintaining distracted disc space while surgery is performed, and allow easier instrument access to the disc space. Further, the devices allow for improved spatial orientation to the disc space through the limited soft tissue access tunnel.

Various embodiments of the distractor device are shown and described herein. In accordance with some or all embodiments, the distractor devices include opposing vertebra-contacting surfaces connected by an intermediate element. The devices may have an “I”, “H”, “C”, “O” or “U” shaped cross-section, or other similar open shapes creating a work portal. Regardless of the shape, positioning of the distractor in an intervertebral space creates a portal providing room for a surgeon to work as well as an improved, direct visualization of the surgical site view within the intervertebral space.

In use, after an annular window is created during an interbody procedure, typically using a wedge-shaped trial, an intervertebral distracting device as disclosed herein is inserted, causing the disc space to distract and increasing the area and volume of access to the space. The distraction device and the portal formed therein permits simultaneous maintenance of distraction and surgical access to the disc. This improves time efficiency of the surgery as well as efficacy of the discectomy. Further it provides increased safety with decreased moving in and out of instruments.

In another embodiment, a distractor includes means for connecting thereto one or more extraspinal soft tissue retractors, to help define a safe “tunnel”.

In another embodiment, the device includes means for connecting an endoscope thereto to enhance intradiscal visualization. This embodiment is useful for example in minimally invasive disc surgeries.

In a further embodiment the intervertebral devices provide a portal which permits navigated placement of intradiscal devices or implants referenced to the annular portal. For example, the device may include one or more grooves and/or flanges which act as guides for proper placement and/or directional insertion of intradiscal devices and/or implants.

### BRIEF DESCRIPTION OF THE DRAWINGS

For the purposes of illustration, there are forms shown in the drawings that are presently preferred, it being understood, however, that the invention is not limited to the precise arrangements and instrumentalities shown.

FIG. 1 is a side cross-sectional view of a wedge-type trial of the prior art;

FIG. 2 is a front view of an intervertebral distractor according to an embodiment of the present disclosure;

FIG. 3 is a front view of an intervertebral distractor according to an embodiment of the present disclosure;

FIG. 4 is a front view of an intervertebral distractor according to an embodiment of the present disclosure;

FIG. 5 is a lateral view of a section of vertebrae and disc;

FIG. 6 is a lateral view of an intervertebral distractor according to an embodiment of the present disclosure positioned in an intervertebral space;

FIG. 7 is an anterior-posterior view of an intervertebral distractor according to an embodiment of the present disclosure positioned in an intervertebral space;

FIG. 8 is a perspective view of an intervertebral distractor according to an embodiment of the present disclosure;

FIG. 9 is a perspective view of an intervertebral distractor according to an embodiment of the present disclosure;

FIG. 10 is a perspective view of an intervertebral distractor according to an embodiment of the present disclosure; and

FIG. 11 is an anterior-posterior view of a distracted intervertebral space with an intervertebral distractor positioned in the space according to an embodiment of the present disclosure.

### DETAILED DESCRIPTION OF THE EMBODIMENTS

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that when an element is referred to as being “coupled” or “connected” to another element, it can be directly coupled or connected to the other element or intervening elements may also be present. In contrast, when an element is referred to as being “directly coupled” or “directly connected” to another element, there are no intervening elements present. Like numbers refer to like elements throughout. As used herein the term “and/or” includes any and all combinations of one or more of the associated listed items.

In addition, spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

Well-known functions or constructions may not be described in detail for brevity and/or clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein. Unless otherwise indicated or defined, all technical and scientific terms used herein have the same meaning as commonly understood by one of ordinary skill in the art to which the invention pertains. The terminology used herein is for describing particular embodiments only and is not intended to be limiting.

Embodiments of the present invention are described with reference to the figures. Now referring to FIG. 1, a wedge-type distractor 1 is shown. The wedge distractor 1 of FIG. 1 may be used as is known in the art to distract—or hold apart—adjacent vertebra.

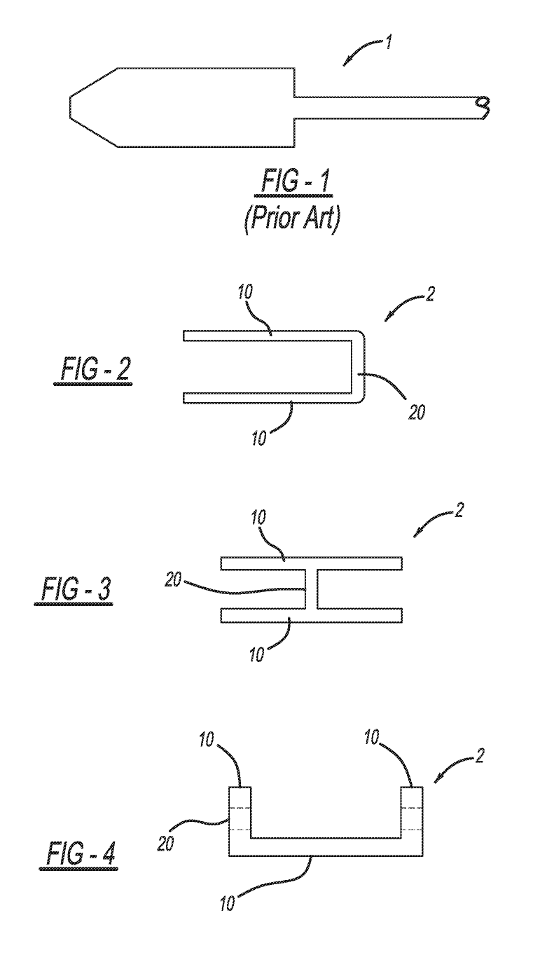

Now referring to FIGS. 2-4 and 8-10, various embodiments of intervertebral distractors 2 in accordance with the present disclosure are shown. Distractors 2 include opposing vertebra-contacting surfaces 10 for contacting and supporting opposing faces of adjacent vertebrae, and one or more intermediate sections 20 connecting the supporting opposing surfaces 10. Surfaces 10 may be textured to enhance surface friction between the surface 10 and the contacted surface of the vertebra.

The dimensions of the opposing surfaces 10 may be from 1 to 40 mm in width, 1 to 60 mm in depth and 1 to 40 mm in length. The dimensions should be adequate to provide sufficient surface area to support and hold adjacent vertebrae in a distracted position. The dimensions of the intermediate section 20 may be from 1 to 10 mm in width, 2 to 60 mm in depth and 2 to 40 mm in length. The dimensions of the intermediate section 20 should be adequate to provide sufficient strength to support and hold adjacent vertebrae in a distracted position, yet be dimensioned to provide maximum possible access for viewing and instrumentation. In one or more embodiments, the intermediate section 20 may be of fixed dimension or expandable, such as by a screw-type expander or the like.

The devices may have an open cross-section in the shape of an “I”, “H”, “C”, “O” or “U”, where the aforementioned two-dimensional cross-section is extended or extruded into the third dimension to create a three-dimensional body of the distractor 2. Regardless of the type font used in the present disclosure, an “I” shape cross-section of the distractor 2 is intended to describe the shape of a common “I-beam”, with top and bottom flanges and a central web serving. The ends of the top and bottom flanges of an “I” section (when rotated 90°) serve as the opposing surfaces 10, whereas the outer sides of an “H” section (when rotated 90°) serve as the opposing surfaces 10 which contact the vertebrae 100,102.

As will be apparent to those skilled in the art, the configuration of each of the distractors 2 in the figures is such that one or more portals are formed when the distractor 2 is positioned in the intervertebral space. The portals are open spaces in which the surgeon can work in an unobstructed fashion with the distractor 2 in position between the vertebrae 100,102. In the “I” and “H” shapes of the distractor 2, two portals are formed, one on each side of the central web of the “I” or “H”. In the “C”, “O” and “U” shapes of the distractor 2, one portal is formed, that being in the center of the “C”, “O” or “U” cross-section. The particular shape or embodiment of the distractor 2 which is chosen by a surgeon depends on where in the intervertebral space the surgeon will be working. For example, if the surgeon needs to work on disc sectioning in the central part of the intervertebral space, then a “C”, “O” or “U” cross-section shape may be most suitable. If the surgeon needs to work on disc sectioning in toward the sides or periphery of the intervertebral space, then an “H” or “I” cross-section shape may be most suitable.

The distractor 2 may be constructed of any material suitable for use in a surgical procedure and capable of withstanding the accompanying loads—where the materials include stainless steel or other metals/alloys, ceramic, plastics and engineered composites such as carbon-fiber. The various shapes of the distractor 2 may be formed by casting, injection molding or extrusion—as appropriate for the material.

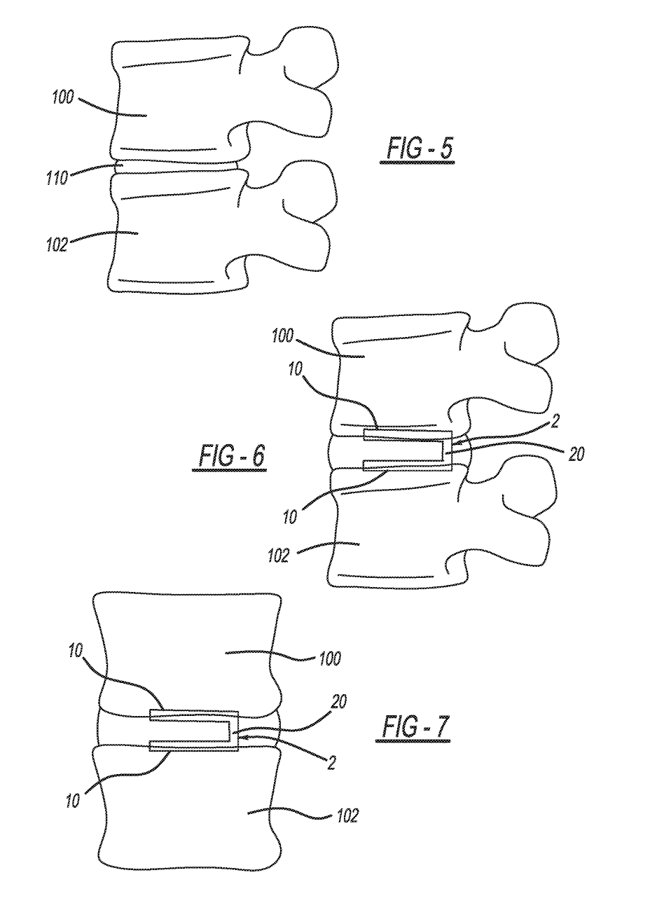

FIG. 5 is a lateral (side) view of two adjacent vertebrae 100 and 102, where a surgical procedure on an intervertebral disc 110 may need to be performed, and the disclosed distractor 2 may be used to increase the intervertebral space. FIG. 6 is a lateral view of the vertebrae 100,102 with the distractor 2 in position and separating the vertebrae 100,102. FIG. 7 is an anterior/posterior (front/rear) view of the vertebrae 100,102 with the distractor 2 in position and separating the vertebrae 100,102. As indicated by FIGS. 6 and 7, the distractor 2 is suitable for use in intervertebral surgical procedures performed from the patient's front (anterior), back (posterior) or side (lateral). Furthermore, the distractor 2 is suitable for use in both open and minimally-invasive intervertebral surgical procedures.

With reference to FIGS. 5-7 and 11, in use, after an annular window is created during an interbody procedure, typically using the wedge-shaped trial 1, an intervertebral distracting device 2 as disclosed herein is inserted, causing the disc space to distract and increasing the area and volume of access to the space. The distraction device 2 and the portal formed therein permits simultaneous maintenance of distraction and surgical access to the disc. This improves time efficiency of the surgery as well as efficacy of the discectomy. Further it provides increased safety with decreased moving in and out of instruments.

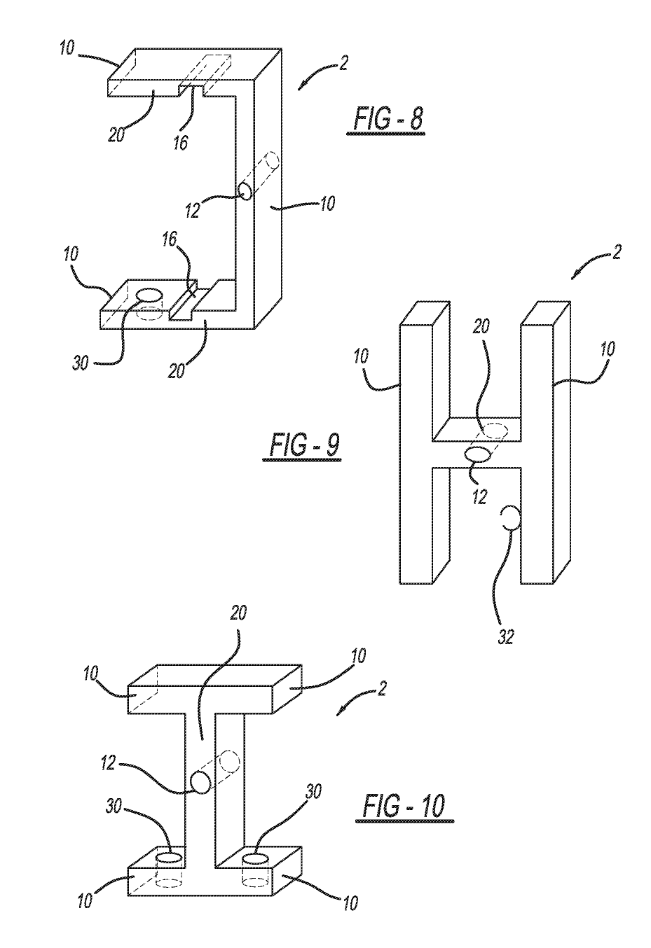

With reference to FIGS. 8 and 9, in some embodiments the distractor 2 includes a means for connecting thereto one or more extraspinal soft tissue retractors, to help define a safe “tunnel” through the soft tissue which must be bypassed in order to access the vertebrae 100,102 where the surgical procedure is taking place. For example, an aperture 12 may be formed in the distractor 2 for receiving and retaining a tissue retractor. The aperture 12 may include a threaded interior to receive external threads of a tissue retractor. Alternatively the aperture 12 may include a quick-release device as known in the art for releasably engaging an end of a tissue retractor. The aperture 12 may also be employed for insertion and manipulation of the distractor 2 in the intervertebral space. In yet a further embodiment, the means for connecting a tissue retractor may include a clip or the like.

In another embodiment the distractor 2 may include a clip, aperture or other means for connecting an endoscope thereto to enhance intradiscal visualization. For example, with reference to FIG. 8 a means for connecting an endoscope is an aperture 30 formed in the distractor 2 which permits passage of a flexible endoscope therethrough. With reference to FIG. 9, the means for connecting an endoscope is a spring clip 32 extending from the distractor 2. The means for connecting an endoscope to the distractor 2 is useful for example in minimally invasive disc surgeries.

The intervertebral distractors 2 provide a portal which permits navigated placement of intradiscal devices or implants referenced to the annular portal. With reference to FIG. 8, in some embodiments the distractor 2 may include one or more guide(s) 16 for proper placement and/or directional insertion of intradiscal devices and/or implants. The guide 16 may be in the form of one or more grooves and/or flanges or the like.

FIG. 10 is a perspective view illustration of an “I”-shaped embodiment of the distractor 2. The distractor 2 of FIG. 10 would be rotated 90° before placement in the intervertebral space, so that the opposing surfaces 10 are in a position to distract (hold apart) the vertebrae. Logical locations are shown for the aperture 12 for a tissue retractor, and the aperture 30 for an endoscope.

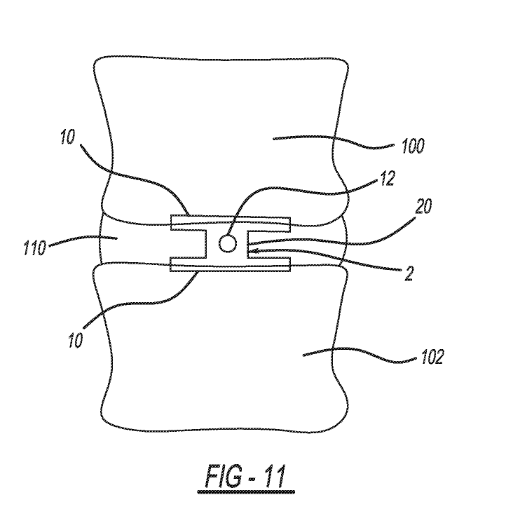

FIG. 11 is an anterior-posterior view illustration of a distracted intervertebral space with an intervertebral distractor 2 positioned in the space. FIG. 11 illustrates an “H”-shaped embodiment of the distractor 2, rotated on its side and positioned between the vertebrae 100,102. The aperture 12 for a tissue retractor is also shown in FIG. 11.

Regardless of the shape, positioning of the distractor 2 in an intervertebral space creates a portal providing room for a surgeon to work as well as an improved, direct visualization of the surgical site view within the intervertebral space. The devices 2 provide a portal for navigation, alignment and access of various tools such but not limited to chisels, saws, osteotomes, etc., for removal of soft or hard tissue.

Although the devices and systems of the present disclosure have been described with reference to exemplary embodiments thereof, the present disclosure is not limited thereby. Indeed, the exemplary embodiments are implementations of the disclosed systems and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refinements are encompassed within the scope of the present invention.

What is claimed is:

1. A distracting portal device for placement between two adjacent vertebrae during an intervertebral surgical procedure, said distracting portal device being a three-dimensional object having a two-dimensional cross-section extended into a third dimension, where the cross-section has a shape which defines one or two portal openings, where each portal opening creates a space between the vertebrae into which a surgeon has clear access, and where opposing top and bottom surfaces of the portal device are configured for contact with and distraction of the two adjacent vertebrae.

2. The device of claim 1 wherein the surgical procedure is a discectomy.

3. The device of claim 1 wherein a distance between the top and bottom surfaces defines a height which is adjustable after the device is in place between the two adjacent vertebrae.

4. The device of claim 1 further comprising means for connecting a soft tissue retractor to the portal device, where the soft tissue retractor creates a tunnel through which surgical tools can reach into the one or two portal openings.

5. The device of claim 4 wherein the means for connecting a soft tissue retractor includes an aperture in the portal device, said aperture being aligned parallel to a line of sight of the one or two portal openings.

6. The device of claim 5 wherein the aperture is threaded to receive the soft tissue retractor or includes a quick-connect coupling for attachment of the soft tissue retractor.

7. The device of claim 1 further comprising means for coupling an endoscope to the portal device, where the endoscope provides improved visualization of items within the space created by the one or two portal openings.

8. The device of claim 7 wherein the means for coupling an endoscope includes an aperture in the portal device through which the endoscope may be passed, where the aperture passes through a wall of the portal device such that neither opening of the aperture is obstructed by the vertebrae.

9. The device of claim 7 wherein the means for coupling an endoscope includes a clip attached to the portal device to which the endoscope may be removably connected.

10. The device of claim 1 further comprising a guide for directional insertion and placement of an implant device in the space created by the one or two portal openings.

11. The device of claim 1 wherein the opposing top and bottom surfaces are textured to enhance friction between the top and bottom surfaces of the portal device and the vertebrae.

12. The device of claim 1 wherein the portal device is selected from a family having a single portal opening and having a cross-section resembling one of the letters “C”, “O” or “U”, or a family having two portal openings and having a cross-section resembling one of the letters “H” or “I”.

13. The device of claim 1 wherein the portal device is a single-piece unit composed of stainless steel, ceramic or a reinforced polymer material.

14. An apparatus for use in a discectomy surgical procedure, said apparatus comprising:
a distracting portal device for placement between two adjacent vertebrae during the discectomy, said distracting portal device being a three-dimensional object having a two-dimensional cross-section extended into a third dimension, where the cross-section has a shape which defines one or two portal openings, where each portal opening creates a space between the vertebrae into which a surgeon has clear access, and where opposing top and bottom surfaces of the portal device are configured for contact with and distraction of the two adjacent vertebrae;
a soft tissue retractor coupleable to the portal device, said soft tissue retractor being used to create a tunnel through which surgical tools can reach into the one or two portal openings; and
an endoscope coupleable to the portal device, said endoscope being used to provide improved visualization of items within the one or two portal openings.

15. The apparatus of claim 14 wherein the portal device is selected from a family having a single portal opening and having a cross-section resembling one of the letters “C”, “O” or “U”, or a family having two portal openings and having a cross-section resembling one of the letters “H” or “I”.

16. The apparatus of claim 14 wherein the soft tissue retractor connects to an aperture in the portal device, said aperture being aligned parallel to a line of sight of the one or two portal openings, where the aperture is threaded to receive the soft tissue retractor or includes a quick-connect coupling for attachment of the soft tissue retractor.

17. The apparatus of claim 14 wherein the endoscope is routed through an aperture in the portal device, where the aperture passes through a wall of the portal device such that neither opening of the aperture is obstructed by the vertebrae.

18. The apparatus of claim 14 wherein the opposing top and bottom surfaces of the portal device are textured to enhance friction between the top and bottom surfaces of the portal device and the vertebrae.

19. A method for performing an intervertebral surgical procedure, said method comprising:
distracting, using a trial-wedge device, two adjacent vertebrae to create a distracted intervertebral space;
inserting a distracting portal device into the distracted intervertebral space, where the distracting portal device is a three-dimensional object having a two-dimensional cross-section extended into a third dimension, and where the cross-section has a shape which defines one or two portal openings, where each portal opening creates a space between the vertebrae into which a surgeon has clear access;
using a soft tissue retractor coupled to the portal device to create a tunnel through which surgical tools can reach into the one or two portal openings;
using an endoscope coupled to the portal device to provide improved visualization of items within the one or two portal openings; and
performing the surgical procedure in the space created by the one or two portal openings.

20. The method of claim 19 wherein the portal device is selected from a family having a single portal opening and having a cross-section resembling one of the letters “C”, “O” or “U”, or a family having two portal openings and having a cross-section resembling one of the letters “H” or “I”.
